(19) United States
(12) Patent Application Publication
(10) Pub. No.: US 2015/0053223 A1
(43) Pub. Date: Feb. 26, 2015

(54) HANDY PEDICURE DEVICE

(71) Applicant: Dean Bingham, Las Vegas, NV (US)

(72) Inventor: Dean Bingham, Las Vegas, NV (US)

(21) Appl. No.: 13/972,174

(22) Filed: Aug. 21, 2013

(51) Int. Cl. A61B 17/54

(52) U.S. Cl. CPC A61B 17/54
USPC 132/76.4

(57) ABSTRACT

A callous removal device including: a strap, where said strap includes an outside surface and an inside surface; a first handle attached to a first end of the strap; and a second handle attached to second end of the strap. The inside surface may include aluminum oxide sandpaper and the outside surface includes cloth.

### BACKGROUND OF THE INVENTION

1. Field of Invention

The present invention relates to a multi-purpose pedicure device to assist in the removal of callous in the foot of a user.

2. Description of Related Art

Many individuals require maintenance of their feet in terms of callouses, corns and nail clipping. Some people take care of this personal hygiene maintenance by going to a boutique for a pedicure by a professional. Others engage into self-maintenance and take care of their pedicure needs on their own. The advantages of self-pedicures include lower cost and flexible timing, however the individual performing the self-pedicure must have adequate equipment and dexterity in order to perform the pedicure. Many times some people may have physical limitations that make it difficult to remove callous or clip toes and to perform other functions related to a pedicure. The removal of callous many times means the use of a file and movement of the file over the surface of the foot in a particular area. This task may be difficult for some people with certain physical limitations. Consequently, it would be advantageous to have a device that could be used during a self-pedicure for callous removal.

### SUMMARY OF THE INVENTION

The present invention relates to a callous removal device comprising: a strap, where said strap includes an outside surface and an inside surface; a first handle attached to a first end of the strap; and a second handle attached to second end of the strap. The inside surface may include aluminum oxide sandpaper and the outside surface includes cloth.

### BRIEF DESCRIPTION OF DRAWINGS

FIG. 1 depicts a callous removing device in accordance with the present invention.

### DETAILED DESCRIPTION

The present invention relates to a device for callous removal that incorporates the use of handles that attach to an easy-to-use pedicure strap. The strap has a predetermined width and one side of the strap includes waterproof aluminum oxide sandpaper. The strap is cloth in nature and therefore has a cloth surface on the opposite side of the aluminum oxide sandpaper. During use an individual grasps both handles and then places the strap across the surface of their foot. By using the strap and the handles, the user may easily move the strap in a back-and-forth direction across the surface of the individual's foot. Sandpaper allows for the removal of the user's callous in an easy and effective manner. This callous removal device of the present invention allows for users to easily reach all areas of their foot and therefore is an advantageous device to use for self-pedicures.

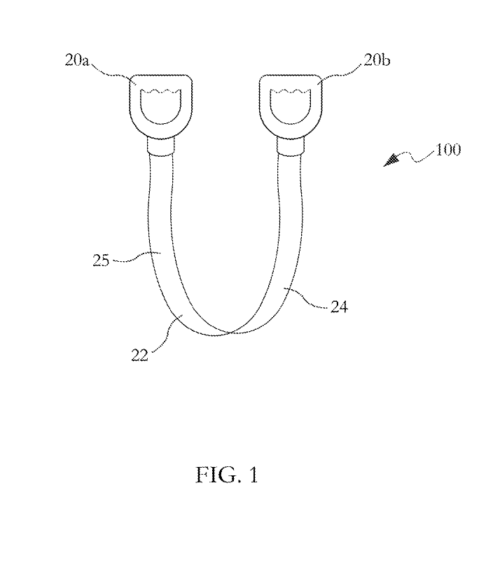

With respect to FIG. 1 a callous removing device 100 is depicted. The callous removing device 100 includes a first handle 20a and a second handle 20b. The first handle is attached to the first end of a strap 25. The second handle 20b is attached to the second end of strap 25. The strap 25 in one particular embodiment may be made of a cloth material where one side of the cloth material includes aluminum oxide sandpaper. The outside surface 22 of the strap is cloth material. The inside surface 24 is coated with the aluminum oxide sandpaper for placement against the outer surface of the user's foot. Other suitable materials for callous removal may also be implemented for the removal of callous.

The easy-to-use callous removal 100 of the present invention allows the user to grasp handles 20a, 20b and move the inside surface 24 across the surface of the foot for removal of callouses that may exist on the skin. Not only is the callous removal 100 easy to use it is very effective and quickly removes callous in a quick and efficient manner. In one particular embodiment the strap may be up to 30 inches in length and about 1½ inches in width. This use of the strap eliminates the need to bend over or to maneuver a person's foot during use.

What is claimed is:

1. A callous removal device comprising:
a. a strap, where said strap includes an outside surface and an inside surface;
b. a first handle attached to a first end of the strap; and
c. a second handle attached to second end of the strap.

2. The callous removal device according to claim 1, where the inside surface includes aluminum oxide sandpaper and the outside surface includes cloth.

3. The callous removal device according to claim 1, where the strap is made of cloth.

4. The callous removal device according to claim 1, where the strap is about 30 inches in length and about 1.5 inches in width.
